(19) United States
(12) Patent Application Publication
(10) Pub. No.: US 2013/0344560 A1
(43) Pub. Date: Dec. 26, 2013

(54) Method for localized photo-irradiation of biological tissues to stimulate tissue regeneration or repair

(71) Applicants: Jon Weston, Highlands Ranch, CO (US); Sherry Fox, Parker, CO (US)

(72) Inventors: Jon Weston, Highlands Ranch, CO (US); Sherry Fox, Parker, CO (US)

(21) Appl. No.: 12/082,585

(22) Filed: Apr. 11, 2008

Related U.S. Application Data
(60) Provisional application No. 60/926,795, filed on Apr. 30, 2007.

(51) Int. Cl. C12N 13/00

(52) U.S. Cl. CPC C12N 13/00
USPC 435/173.8

(57) ABSTRACT

This invention relates to a method of delivering light energy to biological tissues for the acceleration of healing of damaged or diseased tissues or regeneration of such tissues. More particularly, the present invention relates to applying various wavelengths of light to articular and non-articular joints, alone, or in conjunction with techniques for restoring or regenerating cartilage, ligament and/or tendons whether in-vitro, in-situ or in-vivo. The present invention also extends to application of photo-irradiation to stimulate enhanced proliferation and site-dependent differentiation of stem cells into mature cells and to stimulate full functioning of mature cells involved in tissue repairs for regeneration of tendons, ligaments, cartilage, bone or muscle, depending on the type of tissue with which the stem cells come into contact.

this non-provisional patent application claims benefit to the following provisional U.S. patent application U.S. 60/926,795 filed Apr. 13, 2007

### BACKGROUND OF INVENTION

Major orthopedic diseases affect a large proportion of people. Active lifestyles and increasing participation in sports contributes to the incidence of tendon and ligament damage. As people age they become more susceptible to such injuries. In addition, arthritis is a nearly universal condition as people age. Unfortunately natural body repair mechanisms also break down, to an increasing extent, with age. Doctors are constantly searching for better techniques to ensure faster and more durable repair of orthopedic injuries and arthritis. Many of the techniques exhibit higher failure rates with age. Additionally arthritis is generally regarded as a progressive and incurable condition. Often patients are left with the choices of pain and severe functional impairment or complete joint replacement with artificial joints.

Photo-Irradiation Effects On Living Cells

It is well recognized that the application of artificially-created light to tissue may achieve general therapeutic effects, notably, pain relief. The application of light to tissue and blood has the effect of influencing the localized release of nitric oxide (NO), thereby stimulating vasodilation. Studies have demonstrated infrared photochemical generation of nitric oxide by two-photon excitation of precursor molecules such as porphyrin complexes.' Studies of aortic tissues demonstrated the presence of a chemical substance termed “endothelium-derived relaxing factor” (EDRF). This substance was subsequently revealed to be nitric oxide.²

There is evidence that photo-irradiation stimulated release of nitric oxide increases lymphatic circulation by virtue of an increase in the diameter of the lymphatic vessels, not just by increased flow rates within the vessel at an unchanged diameter. This diameter increase, if definitively present, would also explain a facilitated process for removal of debris and larger protein cells passing out of traumatized areas that is additionally stimulated by the use of infrared light therapy.³ Photo-irradiation also has been shown to act to stimulate mitochondria ATP which increases cellular and circulatory motility as well as directly influencing lymphatic flow. It also promotes increased permeability in interstitial tissue and facial layers reducing stagnation and blockage.⁴

Additionally, researchers have proposed a chain of molecular events triggered by visible light irradiation. In this model, light is absorbed by cytochrome components of cellular respiratory chains, stimulating oxidation of Nicotinamide adenine dinucleotide (NAD) which alters the redox potential of mitochondria and cytoplasm in cells. This change in redox potential alters membrane permeability and calcium channels, affecting the levels of cyclic nucleotides which modulate DNA and RNA synthesis, in turn, affecting cell proliferation.⁵ Further evidence points to infrared wavelengths interceding further down the cell respiratory chain, directly affecting the calcium channels with the same downstream effects on DNA and RNA synthesis and cell proliferation.⁶

Photo-Irradiation and Arthritis

There is some evidence that photo-irradiation may affect some of the underlying components and mechanisms of arthritis. This might be achieved through stimulated release of the chemical intermediary nitric oxide which acts to signal cartilage repair through modulation and proliferation of chondrocytes or the precursor cells to chondrocytes. Alternatively, there may be a direct photo-stimulatory effect on chondrocytes or precursor cells to chondrocytes via the respiratory chain or through direct membrane permeability modulation.

Characterization of Arthritis

Arthritis is a degenerative condition affecting the integrity of the cartilage buffer between bones in articular and non-articular joints. Arthritis is the leading cause of disability in people older than fifty-five years. The two main forms of arthritis are Osteoarthritis (OA), resulting from cartilage wear and tear or trauma, and Rheumatoid arthritis (RA), an autoimmune disease. Several underlying mechanisms of arthritis may be affected by light therapy, including inflammation, collagen and proteoglycan formation, and chondrocyte growth and proliferation, and extracellular matrix protein formation.

Cartilage is composed of collagenous fibers and/or elastic fibers, and cells called chondrocvtes, all of which are embedded in a firm gel-like ground substance called the matrix. Cartilage is avascular (contains no blood vessels) and nutrients are diffused through the matrix. Cartilage serves several functions, including providing a framework upon which bone deposition can begin and supplying smooth surfaces for the movement of articulating bones. There are three main types of cartilage: hyaline, elastic and fibrocartilage. Within articular cartilage, Hyaline and Fibrocartilage are the most important.

Types of Cartilage

Hyaline cartilage is the most abundant type of cartilage. The name hyaline is derived from the Greek word hyalos, meaning glass. This refers to the translucent matrix or ground substance. It is avascular hyaline cartilage that is made predominantly of type II collagen. Hyaline cartilage is found lining bones in joints (articular cartilage or, commonly, gristle). It can withstand tremendous compressive force, needed in a weight-bearing joint.

Fibrocartilage (also called white cartilage) is a specialized type of cartilage found in areas requiring tough support or great tensile strength, such as intervertebral discs and at sites connecting tendons or ligaments to bones (e.g., meniscus). There is rarely any clear line of demarcation between fibrocartilage and the neighboring hyaline cartilage or connective tissue. In addition to the type II collagen found in hyaline and elastic cartilage, fibrocartilage contains type I collagen that forms fiber bundles seen under the light microscope. When the hyaline cartilage at the end of long bones such as the femur is damaged, it is often replaced with fibrocartilage, which does not withstand weight-bearing forces as well.

Cartilage is composed of 4% chondrocytes and 96% extracellular matrix. Extracellular matrix is composed of:

Type II collagen, a major support structure (Types I and III also present in smaller amounts)

Proteoglycans, long fibrous chains, chiefly aggrecan. These are configured as globules, encased in the matrix by a mesh-like limiting lattice of Type II collagen. They are hydrophilic (absorbing 30 to 50 times their dry weight) and continually expand—contained by the lattice network of Type II collagen—to provide the shock-absorbing qualities of cartilage.

Glycosaminoglycan chains, composed of keratin sulfate and chondroitin sulfate.

Interstitial fluid, containing chiefly water and a host of proteins.

Chondrocytes balance the breakdown and repair processes of cartilage. They differ from other animal cells in that they have no blood supply, no lymphatics, and lack access to nerves. Joint movement and compression cause flows within the matrix that move diffused nutrients and stimulates the breakdown and repair factors.

Cartilage Metabolism: Promotional and Degradation Factors

As with many body systems, cartilage is maintained by a balance of tissue promotion and degradation factors. Promotional factors include aggrecan and collagen formation and “tissue inhibitor of metalloproteinases” (TIMP). Other pro-cartilage factors include bone growth factors, which have a role in the preservation of the cartilage matrix. These include bone morphogenetic proteins, insulin-like growth factors, hepatocyte growth factor, basic fibroblast growth factor, transforming growth factor beta, and Stress Proteins (also known as Heat Shock Proteins). What these pro-cartilage factors have in common is that they operate directly on stem cells, which are critical to new cartilage formation.

Degradation factors in cartilage include matrix metalloproteinase (MMP) enzymes, aggrecanases, collagenases, activators of MMPs and nitric oxide (inducible form). Within the cartilage matrix, inducible nitric oxide plays an opposite role to that of endothelial nitric oxide found in well-vascularized tissues where it functions as a critical signaling factor for tissue repair. This contradistinction can be seen in other organ systems⁷.

Inducible vs Endothelial Nitric Oxide: Effects in Cartilage

Zhou, et al, examined renal glomerular thrombotic microangiopathy (TMA) and associated levels of inducible nitric oxide synthase (iNOS) and endothelial nitric oxide synthase (eNOS). The investigators found administration of E. coli endotoxin leads to a sustained fall in renal eNOS expression and concomitant rise in iNOS expression both in vivo and in vitro. The associated decline in intrarenal endothelial NO production/availability may result in renal vasoconstriction and a hypercoagulative state, which may contribute to the pathogenesis of endotoxin-induced TMA.⁸

The effects of inducible nitric oxide vs endothelial nitric oxide can be observed in cartilage as well. Cartilage contains mostly the iNOS isoform so it only produces high, damaging levels of NO in disease states, and only when there is preceding injury or infection. The goal would be to suppress iNOS activity. Low levels of NO from sodium nitro prusside or other “physiologic” nitric oxide donors suppress the activity of iNOS. It is believed that photo-irradiation releases NO from endothelial cells and red blood cells (RBCs) at the site of application. And if this is a damaged joint, the iNOS activity sustaining the production of very high levels of inducible NO, will be reduced through the local inhibitory action of the small increase in physiologic endothelial NO concentrations (some 100 to 1000 times less than that produced by iNOS) from adjacent endothelial cells and RBCs. Further cartilage damage would theoretically be minimized, swelling/inflammation reduced and pain would be reduced.

Because infrared light both stimulates endothelial nitric oxide release and directly modulates cells through the membrane calcium channels, (and visible wavelengths modulate cells through upstream cytochrome photo accepters), there may be multiple mechanisms by which photo-irradiation affects cartilage and other tissues.

Arthritis and Cartilage Degradation

A shift in the balance toward pro-inflammatory and cartilage degradation factors leads to OA. In primary OA this results from age-associated “wear and tear” marked partly by shortened collagen and proteoglycan chains and attenuated chondrocyte viability, and partly from a negative spiral of inflammatory reactions resulting in matrix damage. In secondary OA, trauma or disease initiates cartilage damage which persists over time and may trigger the inflammatory spiral. Underlying both forms of OA is the slow repair response in typically observed in cartilage.

Cartilage and Connective Tissue Repair Mechanisms

Studies are revealing that cartilage repair (above and beyond normal maintenance) is achieved through pluripotent progenitor cells or stem cells. Such progenitor cells may subsequently differentiate into any number of types of cells, depending on those in which they are in direct physical contact. This phenomenon is described as “site dependent differentiation.” If the progenitor cells are in contact with hyaline cartilage, they may be influenced to differentiate into new hyaline cartilage producing cells (i.e., chondrocytes producing type II collagen), thus filling in lesions or gaps in cartilage with hyaline cartilage. If they are in contact with fibrocartilage, they differentiate into fibrocartilage producing chondrocytes (i.e., producing type I collagen). If in contact with ligament tissue, they differentiate into ligament.

Tissue Repair Arises from Mesenchymal Stem Cells

Cartilage repair arises from mesenchymal cells, which differentiate into cartilaginous cells and extracellular matrix. One study demonstrated through cell radio labeling that cartilage repair is achieved through proliferation and differentiation of mesenchymal (primordial, undifferentiated) stem cells, not from proliferation of extant cartilage chondrocytes. Autoradiography after labeling with 3H-thymidine and 3H-cytidine demonstrated that chondrocytes from the residual adjacent articular cartilage did not participate in the repopulation of the defect. The repair was mediated wholly by the proliferation and differentiation of mesenchymal cells of the marrow. The label, initially taken up by undifferentiated mesenchymal cells, progressively appeared in fibroblasts, osteoblasts, articular chondroblasts, and chondrocytes.⁹ The same phenomenon was seen with anterior cruciate ligament. Adhesion, spreading, proliferation, and collagen matrix production of human bone marrow stromal cells (BMSCs) on an RGD-modified silk matrix was studied. In the presence of ligament tissue bone marrow cells grew and differentiated into ligament cells on the silk matrix.¹⁰

There are a small number of surface dwelling stem cells in articular cartilage which may be recruited to engraft into cartilage lesions. A study employing isolation of an articular cartilage progenitor cell from the surface zone of articular cartilage using differential adhesion to fibronectin suggests that the in-vivo source of the mesenchymal, or progenitor cells for cartilage lesion repair, is from the surface of the articular cartilage itself.¹¹

Another study points out that mesenchymal stem cells (MSC) proliferate, differentiate, engraft and interface well with adjacent tissues (normal cartilage, bone) and form hyaline-like tissue. This suggests a pathway by which a method that promotes cell growth and proliferation (such as photo-irradiation) might lead to formation of new site-specific cartilage in lesions. Stimulation of MSCs in proximal contact with hyaline cartilage tissue to differentiate and proliferate into chondrocytes would be expected to yield Type II collagen resulting in hyaline formation.¹² Likewise, stimulation of MSCs in proximal contact with fibrocartilage might be expected to result in formation of new fibrocartilage tissue.

Fibrocartilage is the primary type in spinal discs. A recent study by Sakai and colleagues further demonstrates the ability of non-differentiated mesenchymal cells to expand and differentiate into a number of different joint space cells, as influenced by physical contact with native mature cells (site dependent differentiation). In this case, the MSC cells were transplanted into degenerative discs and differentiate into cells expressing a number of key cell-associated matrix molecules in fibrocartilage. MSCs transplanted to degenerative discs in rabbits proliferated and differentiated into cells expressing some of the major phenotypic characteristics of nucleus pulposus cells, suggesting that these MSCs may have undergone site-dependent differentiation.¹³

Site dependent stem cell differentiation extends even to type and layer of cartilage zone. Chondrocyte type and morphology varies depending on the zone of cartilage in which it is found. Chondrocytes from each zone produce a distinct set of matrix components. Stimulation of these various chondrocytes will yield different components, from surface zone proteoglycan, to midzone type II collagen fibers and the high molecular weight aggregating proteoglycan aggrecan which form hyaline cartilage, to deep zone type X collagen.¹⁴ Additionally, stem cells dwelling peripherally throughout the body, representing the fullest variety of tissue types, serve a critical substrate function for tissue repair and regeneration. This includes satellite cells in muscle, fibroblasts in connective tissue, and osteoblasts in bone. In fact, researchers believe most every tissue in the adult body contains tissue specific stem cells. These may be the basis for repair and regeneration. ¹⁵

However, in clinical observation, cartilage lesions often fill in with less functional fibrocartilage.¹⁶ For example, prior experience with cartilage abrasion procedures has shown that defects fill in with fibrocartilage that is less sturdy than hyaline cartilage.¹⁷

Photo-Irradiation and Proliferation and Differentiation of Stem Cells

A series of studies suggest that photo-irradiation may beneficially affect growth and proliferation of various cell types. One such study demonstrates stimulatory effects of low level light therapy on mesenchymal cells—in this instance, mesenchymal cells that differentiate into osteoblasts, which are bone-forming cells.¹⁸ In a study of in-vitro chondrocyte populations, photo-irradiation of test cell cultures indicated a positive biostimulation effect on cell proliferation with respect to the control group.¹⁹ In a study of skin wound healing, the use of 685-nm (red) laser light or polarized light with a dose of 20 J/cm2 resulted in increased collagen deposition and better organization on healing wounds, and the number of myofibroblast was increased when polarized light is used. This study examined the effects of infrared and polarized light on primitive cell (myofibroblasts) proliferation and differentiation in the tissue healing process, using measures of morphologic and cytochemical expression. Photo-irradiation was associated with increased healing activities by these measures as compared to non-treated controls. Taken together with studies of light stimulation of progenitor cells resulting in differentiation and proliferation in a variety of tissue types (e.g., muscle, dermis, bone), this suggests that there may be a general mechanism of action for light on progenitor cells.²⁰

Low level light therapy has been shown to enhance quality of fibroblast cell cultures in terms of engraftment, colony forming efficiency and clonal growth rates.²¹ A fibroblast is a type of cell that synthesizes and maintains the extracellular matrix of many animal tissues. Fibroblasts provide a structural framework (stroma) for many tissues, and play a critical role in skin wound healing. They are the most common cells of connective tissue. Low energy red and infrared light has been shown to promote proliferation of precursor satellite cells in muscle, similar to precursor mesenchymal cells.²²

Techniques for Cartilage Lesion Repair in Arthritis

There are several techniques currently employed for cartilage lesion repair in arthritis. The simplest and least invasive is to brace the load bearing joint to off-load the site of cartilage wear (typically knees) and prescribe daily walking to stimulate matrix movement and repair. Because such arthritis patients are typically older, their natural repair processes are slowed. Such patients may consume nutritional supplements such as glucosamine and chondroitin sulfate in an attempt to enhance the availability of these two cartilage matrix components. Again, clinical observation shows that repairs, when they occur typically are characterized by fibrocartilage in-filing of lesions rather than hyaline cartilage. The number of available surface-dwelling MSCs and the ability to stimulate proliferation and differentiation may be curtailed by age associated declines in tissue repair.

Another non-invasive technique attempting to stimulate cartilage re-growth is the application of electrical stimulation to the joints. A multi-year study showed electrical stimulation delayed knee replacement surgery compared to untreated patients.²³

More invasive techniques rely on methods of presenting chondrocytes or stem cells to the lesion or implanting whole plugs of cartilage tissue. Chondrocytes are harvested, either from autologous or allogeneic sources, and grown in-vitro. They are then transplanted to the lesion site using a section of periosteum membrane to hold the cells in place. The hope is that the cells with engraft the lesion.²⁴ Researchers note that the formation of hyaline cartilage in these transplants center on the bony base of the lesion and close to the periosteum flap, both locations where stem cells are likely to reside.

Another transplant technique, called mosaicplasty, involves boring out cylinders of cartilage from healthy areas of a joint and transplanting to the site of the lesion. Results have been mixed.²⁵

Microfracture is a marrow-stimulation technique in which the lesion is exposed to marrow-derived mesenchymal stem cells. These cells presumably populate the fibrin clot that forms at the defect site following the microfracture procedure in which small holes are punctured into the underlying bone at the site of the lesion and bone marrow stem cells leak out.²⁶ The fibrin “super clots” can in-fill debrided cartilage lesions, leading to new cartilage formation, or anchor ligament implants. In the case of ligaments (for example, anterior cruciate [ACL], posterior cruciate [MCL] and others), a separated ligament is reattached, or a transplanted ligament (either autologous or allogeneic in origin) is attached. In either case the superciot acts as a rich substrate material which site-dependently differentiates into ligament cells and forms a stronger attachment and does so faster than in absence of such stem cells. Two limitations on this technique have been observed. First, in the case of cartilage lesions, often fibrocartilage form in articular lesions rather than hyaline. This provides a less functional repair than if hyaline cartilage formed. Second, in patients over 40 years old, it has been observed that microfracture technique is less effective, whether in ligament repair or cartilage repair. Photo-irradiation can be applied post surgically to enhance proliferation, differentiation and function.²⁷

Other Components of Cartilage and Repair

Photo-irradiation has been shown to induce mucopolysaccharide, which plays a role in improved histopathological findings within arthritic cartilage. The study concluded that the densities of mucopolysaccharide in treated rats increased upon complete (photo-irradiation) treatment more than those of the controls, which is closely related with the improvement in histopathological findings.²⁸ Another study focused on articular chondrocytes and demonstrated that photo-irradiation stimulated growth and secretion of extracellular matrix. In this study, measurements of the products of mid-zone chondrocytes reveal biophotostimulation of these mid-zone chondrocytes.²⁹

Other markers of cartilage (and general tissue) healing include Stress Protein release, leading to improved cartilage healing. Stress Proteins appear when the cell is under stress. They also occur under non-stressful conditions, simply “monitoring” the cell's proteins. Some examples of their role as “monitors” are that they carry old proteins to the cell's “recycling bin” and they help newly synthesized proteins fold properly. These activities are part of a cell's own repair system, called the “cellular stress response.” In this case, photo-irradiation upregulated stress proteins, which is an indicator of cartilage repair processes at work.³⁰

Recent work points out evidence of an inflammatory component to the progression of OA. Essential inflammatory cytokines, such as IL-1 and TNF- are involved initiating a vicious cycle of catabolic and degradative events in cartilage, mediated by metalloproteinases, which degrade cartilage extracellular matrix. The role of inflammation in the pathophysiology and progression of early osteoarthritis is supported further by the observation that C-reactive protein levels are raised in women with early knee osteoarthritis and higher levels predict those whose disease will progress. The synovium from osteoarthritis joints stains for IL-1 and TNF-.³¹ A link between photo-irradiation and anti-inflammatory activity has been observed at the transcriptional level. One key pro-inflammatory molecule is prostaglandin E2 (PGE2). It is synthesized via the cycloxygenase-2 enzymatic pathway. Evidence shows photo-irradiation resulted in down-regulation of prostaglandin E2 at the RNA transcriptional level. Photo-irradiation significantly inhibited PGE2 production in a dose-dependent manner, which led to a reduction of COX-2 mRNA levels.³² Other pro-inflammatory factors have also been shown to be reduced by the application of photo-irradiation. Irradiation with linear polarized infrared light suppressed Interleukin-1 beta-induced (IL-1 β) expression of IL-8 mRNA and, correspondingly, the synthesis and release of IL-8 protein in rheumatoid fibroblast-like synbviocytes cells. This anti-inflammatory effect was equivalent to that obtained with the glucocorticoid dexamethasone. Likewise, irradiation suppressed the IL-1 beta-induced expression of mRNA for pro-inflammatory factors RANTES and GROalpha.³³ Importantly, interleukin-1 beta (IL-1 β), is a key regulator of cartilage degradation. Suppression of IL-1 β should promote cartilage integrity.

Photo-irradiation can demonstrate biological effects at a number of wavelengths and energies, either singly (monochromatic) or in combinations of wavelengths (polychromatic). Abergel and coworkers found that the irradiation of fibroblasts in culture either at 633 nm or at 904 nm stimulated the synthesis of collagen.³⁴

The photo-irradiation may be delivered from any number of sources, including incandescent, light emitting diodes, super luminous diodes, and laser. Experts in photo-biology conclude that lasers are just convenient machines that produce radiation. It is the radiation that produces the photobiological and/or photophysical effects and therapeutic gains, not the machines, and that radiation must be absorbed to produce a chemical or physical change, which results in a biological response.^{35,36} Additionally, an adequate energy dose is required to see the biostimulatory effects. A survey of studies shows that required doses to elicit a biostimulatory response range between 1 joule/cm², and 20 joules/cm², as often as every four hours.³⁷ Some of these studies point to doses even higher—some running as high as 2,700 joules/cm².

Summary of Cartilage and Connective Tissue Repair Components and Factors

Cartilage is a complex living tissue with special challenges in terms of metabolism and repair. There are many components and factors involved in both maintenance and repair mechanisms. A treatment modality that can positively affect some or all of these interacting components may result in useful clinical outcomes for patients who suffer from arthritic diseases. Likewise, as mesenchymal stem cells site-dependently differentiate into any number of tissue types they come into contact with (e.g., surface zone, mid zone and deep zone cartilage; ligament, and bone), the stimulatory effects of photo-irradiation may aid in differentiation, proliferation and engraftment of these new tissues.

### SUMMARY OF INVENTION

The present invention offers a method for stimulating biological regeneration and tissue repair whereby photo-irradiation with wavelengths of both visible and invisible light is used to stimulate growth and site dependent differentiation of:

a) Native dwelling progenitor stem cells found in the surface layers of cartilage

b) Transplanted stem cells or chondrocytes

c) Mesenchymal progenitor or stem cells associated with periosteum flaps or the base of debrided cartilage lesions

d) Transplanted full-thickness cartilage cylinders

e) Mesenchymal progenitor or stem cells drawn from inside the bone via techniques such as “microfracture.”

These stem cells may enhance repair or regeneration of tendons, ligaments, cartilage, bone or muscle, depending on the type of cell they come into contact with. The action of photo-irradiation enhances the normal repair mechanisms in terms of speed and durability of repair or regeneration. This serves to compensate for age-associated retardation and break down of these repair and regeneration mechanisms.

Further, the photo-irradiation may be delivered from a variety of light sources including incandescent, light emitting diodes, super luminous diodes, and laser. Said photo-irradiation sources are to be held or fixed in close proximity to. the body tissue being treated so as to deliver adequate energy—at least 1 joule/cm², optimally 1-20 joules/cm². Some doses may be delivered as high as 2,700 joules/cm². Cooler operating photo-irradiation sources, such as light emitting diodes, superluminous diodes and lasers may be held or fixed in direct contact or up to 5 cm from the skin surface over the body part being treated. Higher temperature sources such as incandescent bulbs must be held or fixed up at least 10 cm above the surface of the skin covering the body part being treated.

### DETAILED DESCRIPTION OF THE INVENTION

The present invention is based on the finding that tissue regeneration or repair is largely dependent on immature pluripotent cells such as satellite cells in muscle, bone marrow stromal cells, mesenchymal stem cells and other peripheral and tissue specific stem cells. These cells site dependently differentiate into local tissue types, growing into mature functional tissues. It is further based on the observation that photo-irradiation of cells can have a number of stimulatory effects leading to improved proliferation in in-vitro experiments. The findings have applications in treating a wide variety of cells and tissues in-vitro prior to tissue transplantation and in-vivo. The stimulative effects of photo-irradiation can help overcome age, disease and other factors which may retard tissue repair mechanisms in the body.

The application of photo-irradiation can stimulate pluripotent cells from any number of origins. In cartilage, a limited number of stem cells dwell in the surface zone along side mature chondrocytes which function to maintain the cartilage matrix. The mature chondrocytes do not function to fill in or regenerate cartilage lesions. The stem cells provide the basis of tissue regeneration. Photo-irradiation stimulates mature chondrocytes to function more efficiently at maintenance of cartilage matrix while also stimulating surface dwelling stem cells to differentiate and proliferate into mature cartilage tissue. This forms the basis of a treatment to maintain joint and cartilage integrity and to stimulate repair of arthritic lesions. Such therapy would function alone in this respect. Additionally, stem cells dwelling peripherally throughout the body, representing the fullest variety of tissue types, serve a critical substrate function for tissue repair and regeneration. This includes satellite cells in muscle, fibroblasts in connective tissue, and osteoblasts in bone. In fact, researchers believe most every tissue in the adult body contains tissue specific stem cells. These may be the basis for repair and regeneration. Application of photo-irradiation, based on in-vitro experiments, may enhance growth and differentiation of these cells, thereby enhancing tissue repair and regeneration.

Another aspect of this invention is to apply photo-irradiation to down-regulate pro-inflammatory cascades, at the transcriptional level, of the cycloxygenase 2 pathway to suppress prostaglandin E2 production and intervene the inflammatory degradation cycle of cartilage. Cycles of inflammation, leading to degeneration of cartilage components through pro-inflammatory activation of destructive enzymes such as matrix metalloproteinases result in degradation of cartilage. By down regulating inflammation, photo-irradiation may reduce the destructive factors in cartilage, allowing reparative factors to build up cartilage integrity and health.

Another aspect of this invention is to apply photo-irradiation to transplanted cells. Such transplant techniques are commonly used to provide an abundance of source tissue material to repair cartilaginous lesions or to replace ligaments. Such transplanted tissues may consist of cells cultures, discrete pieces or segments of tissues, or may consist of entire tissue parts s in the case of ligament transplants. Furthermore, such transplanted tissues may be of autologous or allogeneic sources. Allogeneic transplants may originate from cadaveric tissue or from live donors. The transplanted cells or tissues may also be manipulated prior to transplant in order to prepare or enhance the tissues for more effective engraftment and growth. Chondrocytes or stem cells are often grown and expanded in-vitro. They are then transplanted to a cartilage lesion and sewn into place with a covering flap of periosteum, a membrane that lines the outer surface of bones. This technique presents two applications for photo-irradiation. First is to treat the in-vitro culture to enhance cell function and growth and cell culture expansion, providing both a maximal population of cells for implantation and to enhance cell functionality and quality to maximize potential for successful engraftment. Second is to photo-irradiate the cell culture in-situ, post-transplant. The transplanted cells are now in proximal contact with local recipient tissue. Photo-irradiation can enhance growth, proliferation, functionality and (in the case of stem cells transplant vs mature chondrocyte transplant) site-dependent differentiation into the appropriate local tissue type.

In the case of discrete tissue segment transplantation, photo-irradiation may enhance engraftment and functionality. Another popular cartilage transplant technique is mosaicplasty, which involves boring out cylinders of cartilage from healthy areas of a joint (either autologous or from an allogeneic donor) and transplanting to the site of a cartilage lesion. Treatment of the cartilage cylinders prior to transplant may prepare the cells in the tissue to function optimally upon transplantation. Post transplantation, application of photo-irradiation may enhance engraftment and functionality of the plugs. Furthermore, in the cases of both cell culture transplant and mosaicplasty, the long term duration and survival of the transplant may be enhanced by photo-irradiation.

In addition, the present invention has application for enhancing transplantation of ligament tissue, improving engraftment and mechanical integrity through stimulation of fibroblasts and other ligament cells.

Another aspect of the present invention is to enhance tissue regeneration techniques that utilize mesenchymal progenitor or stem cells drawn from inside the bone via techniques such as “microfracture” to proliferate and site-dependently differentiate into the normal local cartilaginous tissue type at the site of a cartilage lesion, and to fully function upon reaching cell maturity. The microfracture technique involves puncturing holes into the underlying bone to allow bone marrow stem cells to leak out, forming “super clots” which can in-fill debrided cartilage lesions, leading to new cartilage formation, or to anchor ligament implants. In the case of ligaments (for example, anterior cruciate [ACL], posterior cruciate [MU.] and others), a separated ligament is reattached, or a transplanted ligament (either autologous or allogeneic in origin) is attached. In either case the superclot acts as a rich substrate material which site-dependently differentiates into ligament cells and forms a stronger attachment and does so faster than in absence of such stem cells. Two limitations on this technique have been observed. First, in the case of cartilage lesions, often fibrocartilage form in articular lesions rather than hyaline. This provides a less functional repair than if hyaline cartilage formed. Second, in patients over 40 years old, it has been observed that microfracture technique is less effective, whether in ligament repair or cartilage repair. Photo-irradiation can be applied post surgically to enhance proliferation, differentiation and function. Again, age related retardation of tissue repair and regeneration is a common issue. Photo-irradiation can enhance cell function to partially or wholly overcome this issue.

Another aspect of the present invention is employment of a variety of electromagnetic radiation wavelengths. Studies of photo-biostimulation generally have ranged from 445 nanometers (indigo/blue) to 700 nanometers (red). The most effective wavelengths for stimulating cell proliferation seem to center around red wavelengths (around 630 nanometers) and in the near infrared range (800 to 920 nanometers). There are some additional indications that wavelengths longer than these, ranging up to 50,000 nanometers may have beneficial effects. Different wavelengths may be more effective for one type of tissue over another. This suggests that not only monochromatic wavelengths are indicated for treatments, but also mixtures of light wavelengths (polychromatic) may be useful.

The photo-irradiation may be delivered from any number of sources, including incandescent, light emitting diodes, super luminous diodes, and laser. Experts in photo-biology conclude that lasers are just convenient machines that produce radiation. It is the radiation that produces the photobiological and/or photophysical effects and therapeutic gains, not the machines, and that radiation must be absorbed to produce a chemical or physical change, which results in a biological response. Photo-irradiation produces local biostimulatory responses so the light source must be delivered in close proximity to the treated tissue so as to deliver adequate energy—at least 1 joule/cm², optimally 1-20 joules/cm². Some doses may be delivered as high as 2,700 joules/cm². In the case of incandescent photo-irradiation sources, the light emitting bulb should be held or fixed within 10 cm of the treated body part so as to ensure adequate transmission of energy without burning tissue with associated heat generation. In the case of light emitting diodes or super luminous diodes, the light sources may be held or fixed directly against the skin covering the treated body part, or against a translucent material held or fixed in direct contact with the skin. In the case of low intensity (or cold) laser apparatuses, the device should be held or fixed within 2 to 5 cm of the treated body part to ensure adequate and consistent transmission of energy. Application of photo-irradiation treatment may be made as often as every four hours, thus allowing adequate time for cell respiratory cascades to reset, or for nitric oxide sequestration to replenish.

Another aspect of the present invention is the applicability of photo-irradiation treatment to both animals and humans.

### REFERENCES

¹ Photochemical Production of Nitric Oxide via Two-Photon Excitation with NIR Light. Stephen Wecksler, Alexander Mikhailovsky, and Peter C. Ford; J. AM. CHEM. SOC. 9 VOL. 126, NO. 42, 2004, pp13566-67.

² Nitric Oxide and Its Role in Orthopaedic Disease. C. H. Evans, M. Stefanovic-Racic, J. Lancaster. Clinical Orthopaedics and Related Research number 312. pp275-294. 1995.

³ Bone Stimulation by Low Level Laser—A Theoretical Model for the Effects. Philip Gable, B App Sc P.T. G Dip Sc Res (LLLT) MSc, Australia, Jan Tuner, D.D.S., Sweden.

⁴ ibid

⁵ The Photobiological Basis of Low Level Laser Radiation Therapy. K. C. Smith, Laser Therapy 3, 19-24 (1991).

⁶ ibid 3

⁷ Inducible nitric oxide synthase in human diseases. K-D Kröncke, K Fehsel, and V Kolb-Bachofen. Clin Exp Immunol. 1998 August; 113(2): 147-156.

⁸ Down Regulation of Renal Endothelial Nitric Oxide Synthase Expression in Experimental Glomerular Thrombotic Microangiopathy. Xin J Zhou, et al. Lab Invest 2000, 80:1079-1087.

⁹ Cell origin and differentiation in the repair of full-thickness defects of articular cartilage. Shapiro F, Koide S, Glimcher M J. J Bone Joint Surg Am. 1993 Apr;75(4):532-53.

¹⁰ Sequential Growth Factor Application in Bone Marrow Stromal Cell Ligament Engineering. Jodie E. Moreau, Ph.D. Tissue Engineering. Nov. 1, 2005, 11(11-12): 1887-1897. doi:10.1089/ten.2005.11.1887.

¹¹ The surface of articular cartilage contains a progenitor cell population Gary P. Dowthwaite, Joanna C. Bishop, Samantha N. Redman, Ilyas M. Khan, Paul Rooney, Darrell J. R. Evans, Laura Haughton, Zubeyde Bayram, Sam Boyer, Brian Thomson, Michael S. Wolfe and Charles W. Archer. Journal of Cell Science 117, 889-897 (2004).

¹² Repair of Large Articular Cartilage Defects with Implants of Autologous Mesenchymal Stem Cells Seeded into β-Tricalcium Phosphate in a Sheep Model. Tissue Engineering, Nov. 2004, Vol. 10, No. 11-12: 1818-1829.

¹³ Differentiation of Mesenchymal Stem Cells Transplanted to a Rabbit Degenerative Disc Model: Potential and Limitations for Stem Cell Therapy in Disc Regeneration. Sakai, Daisuke MD et.al. Spine. 30(21):2379-2387, Nov. 1, 2005.

¹⁴ Differentiation of chondrocytes across cartilage zones and the resultant matrix component synthesis. Gary P. Dowthwaite, Joanna C. Bishop, Samantha N. Redman, Ilyas M. Khan, Paul Rooney, Darrell J. R. Evans, Laura Haughton, Zubeyde Bayram, Sam Boyer, Brian Thomson, Michael S. Wolfe and Charles W. Archer. Journal of Cell Science 117, 889-897 (2004)

¹⁵ National Institutes of Health NIH News Press Release. Thursday, Jul. 8, 2004 Contact: Bob Kuska.

¹⁶ The Articular Cartilage After Osteotomy for Medical Gonarthrosis: Biopsies after 2 years in 19 cases. ACTA Orthop. Scandinavica, 63: 413-416, 1992.

¹⁷ Arthroscopically Guided Jamshidi Needle Biopsy of Articular Cartilage: Potential Utility in the Evaluation of Disease Modifying Osteoarthritis Drugs (DMOADS). Nathan Wei, MD. Journal of Applied Research Vol 3 Iss3, 2007. http://jmlappliedresearch.com/articlesVol3Iss3/Wei.htm.

¹⁸ Biostimulation of bone marrow cells with a diode soft laser. Dortbudak 0, Haas R, Mallath-Pokomy G. Clin Oral Implants Res. 2000 Dec:11(6):540-5.

¹⁹ Laser biostimulation of cartilage: in vitro evaluation P. Torricelli, G. Giavaresi, M. Fini, G. A. Guzzardella, G. Morrone, A. Carpi and R. Giardino. Biomedicine & Pharmacotherapy Volume 55, Issue 2, March 2001, Pages 117-120.

²⁰ Polarized Light (400-2000 nm) and Non-ablative Laser (685 nm): A Description of the Wound Healing Process Using Immunohistochemical Analysis. Dr. Antonio Luiz B. Pinheiro, Ph.D., et. al. Photomedicine and Laser Surgery, Oct 2005, Vol. 23, No. 5: 485-492

²¹ Laser therapy accelerates initial attachment and subsequent behaviour of human oral fibroblasts cultured on titanium implant material: A scanning electron microscopic and histomorphometric analysis. Maawan Khadral, et. al. Clinical Oral Implants Research, Volume 16 Issue 2 Page 168-April 2005.

²² Skeletal muscle cell activation by low-energy laser irradiation: A role for the MAPK/ERK pathway. Gavriela Shefer. Journal of Cellular Physiology Volume 187, Issue 1, Pages 73-80, 2001.

²³ Michael A. Mont, MD, et al. Pulsed Electrical Stimulation to Defer TKA in Patients with Knee Osteoarthritis. Orthopedics. October 2006. Vol. 29. No. 10. Pp. 887-892.

²⁴ “Autologous Chondrocyte Implantation Compared with Microfracture in the Knee: A Randomized Trial” by Gunnar Knutsen, MD, et al. by the The Journal of Bone and Joint Surgery, Inc. 2004.

²⁵ Horas U, Pelinkovic D, Herr G et al. Autologous chondrocyte implantation and osteochondral cylinder transplantation in cartilage repair of the knee joint. J Bone Joint Surg 2003; 85(2):185-192.

²⁶ Ibid 22.

²⁷ Is Microfracture of Chondral Defects in the Knee Associated With Different Results in Patients Aged 40 Years or Younger? P. Kreuz. Arthroscopy: The Journal of Arthroscopic & Related Surgery , Volume 22, Issue 11, Pages 1180-1186.

²⁸ Effects of helium-neon laser on the mucopolysaccharide induction in experimental osteoarthritic cartilage. Lin Y S, Huang M H, Chai C Y. Osteoarthritis Cartilage. 2006 Apr; 14(4):377-83. Epub 2005 Dec 13.

²⁹ Effect of low-power He-Ne laser irradiation on rabbit articular chondrocytes in vitro Ya-Li Jia, Zhou-Yi Guo. Lasers Surg. Med. 34:323-328, 2004.

³⁰ Effects of helium-neon laser on levels of stress protein and arthritic histopathology in experimental osteoarthritis. Lin Y S, Huang M H, Chai C Y, Yang R C. American Journal of Physical Medicine & Rehabilitation. 83(10):758-765, October 2004.

³¹ Novel strategies for the treatment of osteoarthritis. Chikanza I.1; Fernandes L. Expert Opinion on Investigational Drugs, Volume 9, Number 7, July 2000, pp. 1499-1510(12).

³² Inhibitory effect of low-level laser irradiation on LPS-stimulated prostaglandin E2 production and cyclooxygenase-2 in human gingival fibroblasts. Sakurai Y, Yamaguchi M, Abiko Y. European Journal of Oral Science 108: 29-34, February 2000.

³³ Anti-inflammatory effect of linear polarized infrared irradiation on interleukin-1beta-induced chemokine production in MH7A rheumatoid synovial cells. Shibata Y, et.al. Lasers Med Sci.; 20(3-4): 109 Dec 13, 2005.

³⁴ Abergel, R. P., Meeker, C. A., Lam, T. S., Dwyer, R. M., Lesavoy, M. A. and Uitto, J. (1984). Control of connective tissue metabolism by lasers: recent developments and future prospects. Journal of the American Academy of Dermatology 11, 1142-1150.

³⁵ The Photobiological Effect of Low Level Laser Radiation Therapy. Laser Therapy, Vol. 3, No. 1, Jan-Mar 1991.

³⁶ Low-Energy Laser Therapy: Controversies and New Research Findings. Jeffrey R. Basford, M.D. Lasers in Surgery and Medicine 9:1-5, Mayo Clinic, Rochester, Minn., 1989

³⁷ A systematic review of low level laser therapy with location-specific doses for pain from joint disorders. Bjordal J M, Couppe C, Chow R T, Tuner J and Ljunggren A E (2003): Australian Journal of Physiotherapy 49: 107-116.

The invention claimed is:

1. A method for stimulating biological regeneration and tissue repair with photo-irradiation whereby light energy in discrete wavelengths, both visible and invisible, is used to stimulate growth and full function of mature cells and to stimulate site dependent differentiation, proliferation and full function of immature cells.

2. A method as defined in claim 1 to stimulate native dwelling progenitor stem cells found in the surface layers of cartilage to engraft in cartilage lesions as a tissue type normally found in that location of the joint.

3. A method as defined in claim 1 to stimulate transplanted stem cells (autologous or allogeneic) in a cartilage lesion to proliferate, site-dependently differentiate into the normal local tissue type, and to fully function upon reaching cell maturity.

4. A method as defined in claim 1 to stimulate transplanted chondrocytes (autologous or allogeneic) in a cartilage lesion to proliferate, site-dependently differentiate into the normal local tissue type, and/or to fully function upon reaching cell maturity.

5. A method as defined in claim 1 to stimulate transplanted cartilage cylinders (autologous or allogeneic) in a cartilage lesion to proliferate, site-dependently differentiate into the normal local tissue type, and/or to fully function upon reaching cell maturity.

6. A method as defined in claim 1 to stimulate mesenchymal progenitor or stem cells associated with periosteum flaps or the base of debrided cartilage lesions to proliferate, site-dependently differentiate into the normal local tissue type, and to fully function upon reaching cell maturity.

7. A method as defined in claim 1 to stimulate mesenchymal progenitor or stem cells drawn from inside the bone via techniques such as “microfracture” to proliferate, site-dependently differentiate into the normal local tissue type, and to fully function upon reaching cell maturity.

8. A method as defined in claim 7 to stimulate mesenchymal progenitor or stem cells drawn from inside the bone via techniques such as “microfracture” to proliferate, site-dependently differentiate into the normal local cartilaginous tissue type at the site of a cartilage lesion, and to fully function upon reaching cell maturity.

9. A method as defined in claim 7 to stimulate mesenchymal progenitor or stem cells drawn from inside the bone via techniques such as “microfracture” to proliferate, site-dependently differentiate into the normal local ligament tissue type at the site of a ligament attachment, and to fully function upon reaching cell maturity.

10. A method to stimulate growth and proliferation of cultured cells in an in-vitro environment for later use in implantation into a cartilage lesion.

11. A method as defined in claim 1 to stimulate function of mature cartilaginous chondrocytes to maintain the extracellular matrix of cartilage through growth factors including aggrecan, “tissue inhibitor of metalloproteinases” (TIMP), bone growth factors (which have a role in the preservation of the cartilage matrix), including bone morphogenetic proteins, insulin-like growth factors, hepatocyte growth factor, basic fibroblast growth factor, transforming growth factor beta, and stress proteins.

12. A method as defined in claim 1 to stimulate function of mature cartilaginous chondrocytes to maintain the extracellular matrix of cartilage through production of functional extracellular matrix components including collagen (Type I and/or Type II), proteoglycans glycosaminoglycan chains, keratin sulfate and chondroitin sulfate.

13. A method to down-regulate pro-inflammatory cascades, at the transcriptional level, of the cycloxygenase 2 pathway to suppress prostaglandin E2 production and intervene the inflammatory degradation cycle of cartilage.

14. A method as defined in claim 1 to stimulate stem cells to enhance repair of tendons, ligaments, cartilage, bone or muscle, depending on the type of cell they come into contact with in a site-dependent manner.

15. A method as defined in claim 1 further comprised of photo-irradiation delivered from a variety of light sources including incandescent, light emitting diodes, super luminous diodes, and laser.

16. A method as defined in claim 15 wherein the energized light sources emit substantially monochromatic light at wavelengths ranging from 445 nanometers to 50,000 nanometers, with a preference for wavelengths ranging from 445 nanometers to 920 nanometers.

17. A method as defined in claim 15 wherein the energized light sources emit polychromatic or mixed light wavelengths ranging from 445 nanometers to 50,000 nanometers.

18. A method as defined in claim 15, further comprising positioning the application surface against or around a joint such as to deliver photo-irradiation doses to the joint capsule in the optimal dose ranges to elicit biostimulatory responses of between 1 joule/cm², and 20 joules/cm², as often as every four hours, with possible doses as high as 2,700 joules/cm².

19. A method as defined in claim 16, applied to humans.

20. A method as defined in claim 16, applied to animals.
